(19) United States
(12) Patent Application Publication
(10) Pub. No.: US 2024/0408404 A1
(43) Pub. Date: Dec. 12, 2024

(54) METHOD AND SYSTEMS FOR OTOLOGICAL HEALTH

(71) Applicants: MARK H. KRIETZMAN, Palos Verdes, CA (US); Renz Wolfgang, Rheinfelden (DE)

(72) Inventors: Mark H. Krietzman, Rancho Palos Verdes, CA (US); Wolfgang Renz, Rheinfelden (DE)

(21) Appl. No.: 18/812,714

(22) Filed: Aug. 22, 2024

Related U.S. Application Data
(63) Continuation-in-part of application No. 18/017,652, filed on Jan. 23, 2023, now Pat. No. 12,097,384.
(60) Provisional application No. 63/161,782, filed on Mar. 16, 2021.
(60) Provisional application No. 63/161,801, filed on Mar. 16, 2021.

(51) Int. Cl. A61N 5/06

(52) U.S. Cl. CPC A61N 5/0603; A61N 2005/0605; A61N 2005/0627

(57) ABSTRACT

Disclosed herein are devices and systems wherein a controller is configured to respond to inputs to deliver light spectrum energy including directing at least one locally effective dose (EDE) or aliquot of energy into an ear canal one or more of increasing circulation in the cochlea vasculature and increasing nitric oxide in the local area adjacent to the cochlea and wherein vasodilation increases in the cochlear microvasculature. Effective doses of light energy are adjusted with a control module.

### CROSS-REFERENCE TO RELATED APPLICATIONS

The present application is Continuation in part of U.S. patent application Ser. No. 18/017,652 filed Jan. 23, 2023 which was a 371 National Stage of International Patent Application No. PCT/US2022/020058, filed Mar. 11, 2022, which claims priority to United States (“U.S.”) provisional patent application 63/161,782 filed Mar. 16, 2021 and United States (“U.S.”) provisional patent application filed 63/161,801 filed Mar. 16, 2021 each of which is hereby incorporated by this reference as if fully set forth herein.

### BACKGROUND

### 1. Field of the Disclosure

The present disclosure relates generally to ontological health.

### RELATED ART

Hearing decline and vision declines are serious quality of life and health problem which leads to additional health issues.

A Johns Hopkins study that tracked hundreds adults for over a decade, and found that even mild hearing loss doubled dementia risk. Moderate loss tripled risk, and people with a severe hearing impairment were five times more likely to develop dementia.

The NIH (National Institute of Health) explains that tinnitus is commonly described as a ringing in the ears, but it also can sound like roaring, clicking, hissing, or buzzing. It may be soft or loud, high pitched or low pitched. You might hear it in either one or both ears.

People who work in noisy environments—such as factory or construction workers, road crews, or even musicians—can develop tinnitus over time when ongoing exposure to noise damages tiny sensory hair cells in the inner ear that help transmit sound to the brain. This is called noise-induced hearing loss.

Service members exposed to bomb blasts can develop tinnitus if the shock wave of the explosion squeezes the skull and damages brain tissue in areas that help process sound. In fact, tinnitus is one of the most common service-related disabilities among veterans returning from Iraq and Afghanistan.

The increased use of portable music players (PMP) and smartphone which play audio through earbuds/over ear headphones has become commonplace and, in some instances, has led to increased hearing loss in children and the general population.

As the inner ear is constantly using energy, blood supply to the ear is particularly important. Controlling this blood supply relies on how well individual cells in a blood vessel can contract and relax to change its width, as this determines how much blood reaches the inner ear. There is evidence that conditions such as diabetes, and, more generally, ageing, damage blood vessels and disrupt the control of blood flow to the ear, which may lead to hearing loss. Exposure to loud noise can impair cochlear microcirculation and cause noise-induced hearing loss. TNF-α signaling has been shown to be activated in such loss and to control artery vasoconstriction that regulates cochlear microcirculation.

It is therefore a desideratum to provide supplements and modalities, which may be combinatorial to protect and improve hearing.

### Disclosure

Disclosed herein are aspects of methods and systems providing pharmaceutical and/or nutraceutical effective doses of energy (EDE), an EDE dose may also be referred to as an aliquot of photodynamic light. Said EDE is to maintain or restore homeostasis to hearing and/or the cochlear microcirculation. Said maintenance and/or restoration is to limit, reverse or slow down hearing degradation and the impact such loss has on quality of life and well-being.

Disclosed herein are aspects of supplements of EDE to help maintain homeostasis for the cochlear microcirculation. Said EDE may also reduce instances of tinnitus, limit, reverse or slow down hearing degradation and onset of dementia. In some instances the supplement is a locally EDE direct in an ear canal. In some instances the EDE directed in an car canal at least one of increases circulation in the cochlea vasculature and increases nitric oxide in the local area of the car canal adjacent to the cochlea. In some instances, optionally, a supplement of gaseous hydrogen or oxyhydrogen may be added into the car canal before, during and after applying the EDE.

In some instances the disclosed methods and device further include placing car coverings over a user's ears and directing the gaseous fluid onto the ear canal. In some instances the gaseous fluid is one of hydrogen and oxyhydrogen. In some instances to scavenge hydrogen and vent hydrogen gas an enclosure configured to vent hydrogen is placed over the car coverings.

In some instances the disclosed methods and device further include placing car coverings over a user's ears and directing the gaseous fluid onto the ear canal and one or more of increasing circulation, increasing nitric oxide, reducing oxidative stress, inflammation, and autophagic stress in the cochlea vasculature and/or in the local area adjacent to the cochlea.

The present disclosure provides aspects of devices, methods and systems to deliver an EDE of photodynamic energy to the car canal with one of lasers and light emitting diodes (LEDs). Said LEDs or lasers are mounted on the device facing inward to the car canal and in signal communication with a controller and a power supply to produce EDE for spectra of at least one of red, long red and near IR. The aliquots of light in predetermined spectra are applied via a controller and the light may be applied in partial allocations via intervals. At least one sensor may be in signal communication with the controller whereby the photodynamic energy, energy or spectra of light over time is measured and said measurement is supplied to the controller which is configured to adjust the light output at least based in part on sensor data.

The present disclosure provides aspects of devices, methods and systems to deliver therapeutically effective amounts of light energy to at least increase circulation in cochlear microvasculature. Aspects of the device include an energy delivery device having a means to emit light energy, a power supply, one or more temperature sensors in thermal communication with the car canal and a microprocessor controller in signal communication with the means to emit light energy, the power supply and the one or more temperature sensors. The controller maybe configured to deliver at least one effective dose or aliquot of light energy into an car canal to produce heat in the car canal; and, the controller may be configured to receive temperature sensor input and to control outputs of energy to limit heat in the car canal. In some instances the controller is configured to receive a user's selected comfort level input to limit heat in the car canal. In some instances said effective dose one or more of increases circulation in the cochlea vasculature, increases vasodilation in the cochlear microvasculature and increases nitric oxide in the local area adjacent to the cochlea. In some instances the effective dose is at least one of red light, long red light, near IR light and blue light. In some instances the light is delivered via sequential modes adjusted by the control module. In some instance the controller is configured to adjust of mode, frequency, duration and intensity of the effective dose by way of sensor input to the control module.

The present disclosure provides aspects of devices, methods and systems to deliver therapeutically effective amounts of light energy to at least increase circulation in cochlear microvasculature. Aspects of the device include an energy delivery device having a means to emit light energy, a power supply, one or more temperature sensors in thermal communication with the car canal and a microprocessor controller in signal communication with the means to emit light energy, the power supply and the one or more temperature sensors. The controller maybe configured to deliver at least one effective dose or aliquot of light energy into an car canal to produce heat in the car canal and, the controller is configured to receive temperature sensor input and to control outputs of energy to limit heat in the ear canal and the controller receives sensor input of one of one or more of decibels, vibration of the car canal structure, frequency and/or duration of sound or noise and the controller decides if a threshold is met for application of the dose or aliquot of light energy. In some instances the controller receives sensor input of one of one or more of decibels, vibration, frequency and/or duration of sound or noise outside the ear canal and, the controller decides if a threshold is met for application of the dose or aliquot of light energy. In some instances one or more sensors provide input to the controller for adjustment of mode, frequency, duration and intensity of the effective dose based on the frequency and/or duration of sound or noise outside the car canal. In some instances the energy delivery device is configured to fit in an over the ear chamber and said chamber positions the light output into the car canal. In some instances energy delivery device is configured to be connected by flexible lines in the over the car chamber.

The present disclosure provides aspects of system to deliver therapeutically effective light energy to at least the cochlear microvasculature including an energy delivery device configured to fit into the car canal of a user having a controller in signal communication with at least a means to emit light energy, a power supply and one or more temperature sensors in thermal communication with the car canal. The controller is configured to cause the energy delivery device to deliver at least one effective dose or aliquot of light energy into an ear canal. In some instances the controller is configured to receive temperature sensor input and to control outputs of energy to limit heat in the car canal. In some instances the controller is configured to receive sensor input of one of one or more of decibels, vibration of the ear canal structure, frequency and/or duration of sound or noise. In some instance the controller decides if a threshold level of one or more of decibels, vibration of the car canal structure, frequency and/or duration of sound or noise is met. In some instances if the threshold level is met the controller cause the application of the dose or aliquot of light energy.

It is appreciated by those skilled in the art that some of the circuits, components, controllers, computing devices, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs”), time-to-frequency conversations, frequency-to-time conversions, database mapping, signal processing steps, coding, modulations, demodulations, etc. The controller devices and smart devices disclosed herein operate with memory and processors whereby code is executed during processes to transform data, the computing devices run on a processor (such as, for example, controller or other processor that is not shown) which may include a central processing unit (“CPU”), digital signal processor (“DSP”), additional memory may be added, application specific integrated circuit (“ASIC”), field programmable gate array (“FPGA”), microprocessor, etc. Alternatively, portions DCA devices may also be or include hardware devices such as RFID, NFC and logic circuitry, a CPU, a DSP, ASIC, FPGA, etc. and may include hardware and software capable of receiving and sending information. Reporting of collected data may be via a cellular network or other internet connection as known in the art.

The present disclosure provides, at least in part, as part of a combinatorial approach which may or may not include compositions comprising at least one vasodilator and a carrier agent. In other instances at least one of intravenous injection of gelatin and corticosteroid to improve transport of fluids without increasing pressure withing the cochlear microcirculatory system.

By “combination” or “in combination with,” it is not intended to imply that the therapy or the therapeutic agents must be administered at the same time and/or formulated for delivery together (e.g., in the same composition), although these methods of delivery are within the scope described herein. The immunomodulator and the second therapeutic agent can be administered concurrently with, prior to, or subsequent to, one or more other additional therapies or therapeutic agents. The agents in the combination can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

### DRAWINGS

The disclosure may be better understood by referring to the following figures. The components in the figures are not necessarily to scale, emphasis instead being placed upon illustrating the principles of the invention. In the figures, like reference numerals designate corresponding parts throughout the different views.

FIG. 1A is an overview of ear anatomy and a disclosed energy device and method to deliver aliquots of energy.

FIG. 1B is a representation of an energy delivery device to deliver aliquots of energy of the disclosure positioned on a subject/user.

FIGS. 1C & 1D are representations of an energy delivery device and sensors to deliver aliquots of energy of the disclosure positioned in a subject/user.

FIG. 1E is a representation of a smart earplug which can deliver energy based on sensor measures to the ear canal.

FIG. 2 is an overview of a wearable energy delivery and sensor device of the disclosure.

FIG. 3A is a component view of aspects of an ear covering wearable energy delivery device and system with the energy deliver integrated into the ear covering and feedback device of the disclosure.

FIG. 3B is a component view of aspects of an ear covering wearable energy delivery system with movable energy deliver device within the ear coverings.

FIG. 4 shows aspects of an electrical control and power system of the disclosed wearable energy delivery and feedback device of the disclosure.

FIG. 5 is an overview of a device to deliver prepackaged topical localized doses of therapeutic agent and/or adjuvants of the disclosure.

FIG. 6 is a flow diagram of aspects of the operation of a device and method to deliver controlled aliquots of energy to a user and to receive feedback device of the disclosure.

FIG. 7 is a flow diagram of aspects of a compliance, use and monitoring method to receive use data and evaluate treatment compliance.

FIG. 8A shows a partial cut-away view of a wearable energy and fluid delivery device of the disclosure.

FIG. 8B shows the device of FIG. 8A as part of a method of delivering energy and fluid to the ear canal.

FIGS. 9A and 9B show a low pressure gaseous fluid delivery and collection system.

In the Figures, like reference numerals designate corresponding parts throughout the different views. All callouts and annotations are hereby incorporated by this reference as if fully set forth herein. All cited references are hereby incorporate by this reference as if fully set forth herein.

It will be understood that various aspects or details of the invention may be changed without departing from the scope of the invention. It is not exhaustive and does not limit the claimed inventions to the precise form disclosed. Furthermore, the foregoing description is for the purpose of illustration only, and not for the purpose of limitation. Modifications and variations are possible in light of the above description or may be acquired from practicing the invention. The claims and their equivalents define the scope of the invention.

### Further Disclosure

Disclosed herein are methodologies and therapies including but not limited to light therapy (photodynamic), localized hyperthermia and chemical. The therapies may be combined or applied separately to support hearing homeostasis, treat hearing loss, mitigate the effect of noise or high decibels on hearing and/or treat tinnitus.

Exposure to blue light affects various bio-physiological and psychological functions of the human body and may be called “bioactive” via circadian stimulation. Many of these effects are beneficial. For instance, a region of what is commonly called the blue wavelength region of light may improve memory performance and cognitive function. Exposure to blue wavelength light during memory consolidation has been shown to improve subsequent delayed memory recall when compared to placebo wavelength light exposure. Additionally, blue wavelength light may decrease blood pressure, increase blood flow. Full body irradiation with blue light has been shown to promote release nitric oxide from the skin into circulating blood.

Red spectra may be called long red within a spectrum of >625 nms to <700 nms with peak wavelengths>640-670 nm and Near-Infrared typical ranges from >700 nms and <1400 nm (with typical peak wavelengths: 850 nm, 940 nm, 1064 nm) and affect bio-physiological functions and are also described herein as “bioactive” they have been associated with improved eye health, skin health, hair growth, and cognitive function. Emissions of Long Red and Near-Infrared may be referred to collectively as Red Bioactive light (RBL). Those of ordinary skill in the art and the skilled artisan will recognize variation is narrow and does not create substantial uncertainty in the terms. Hence the terminology RBL is encompasses the entirety of both long red and near-infrared.

Infrared (IR) LED treatment can penetrate the skin between 5 and 10 mm and has been used to treat wounds, ulcers, recalcitrant lesions, and cutaneous scleroderma. Simulations using LEDs have been carried out for a range of beam widths in the range 1 to 40 mm. The results show that as the beam width is increased there is an increase in the depth of penetration, which can clearly be seen by the use of intensity contours, each representing a 1% fraction of the maximum intensity. The 1% fraction contours were chosen after experimentation with other values for penetration such as 1/e and 1/e² for a clear and clean graphical representation of the photo-distribution matrix. It is shown that the beam divergence as the light passes through the skin model.

“Bioactive Exposure” refers to one or both of RBL and blue light directing at least one of at a biological system which may be a specific organ or any part of the body

Disclosed herein are additional methods and systems, which include but are not limited to, providing bioactive lighting (or energy spectrum) exposure as one of a supplement and therapeutic dose for:

A. Tinnitus caused by one or more of Ménière's disease or exposure to loud noise or explosion.

B. Reducing the effect of tinnitus and in particular tinnitus from excessive noise exposure via reduction of vascular pressure. Red/Near Infrared (R/NIR) light, has been used clinically for wound healing, plastic surgery, chronic joint pain and in the field of dermatology. Many of the effects of R/NIR are related to observed effects on blood flow and angiogenesis, which are driven at least in part through nitric oxide dependent processes.

C. Mitigating the result of excessive noise exposure via reduction of TNF-α and/or enhanced levels of interferon γ.

D. Therapy and/or adjuvant for treating Tinnitus via using LED produced light, including but not limited to RBL, as a local heat source to penetrate tissues surrounding the cochlea and stimulate metabolism in the region localized to the cochlea microcirculatory system.

E. Promoting a homeostatic environment in and around the ear canal including reducing or ameliorating inflammation associated with the ear canal, microvasculature, cochlea microcirculatory, cilia and other and hearing structures therein.

In some instances the system may switch between the two modes. Such a system may also be operated in a mode where both an RBL and a blue light are operated simultaneously or through a rapid switching mode (e.g. pulse width modulation to regulate the apparent intensity of each one). The modified color is bioactive and may be regulated by the control system. In some instances red in non-visible region also referred to as near infrared may be used simultaneously or through a rapid switching mode with RBL and which can provide localized heating of tissue. The light modes may be applied in doses or aliquots based on a lookup table or sensor data or other inputs to the control systems. Such light may be used as a health supplement based controls systems. Application of light may also be one of applied via a predetermined algorithm or via a dynamic control system controlled, at least in part, by physiological measures of the user including but not limited to data harvested from sensors associated with or information about users, such as one or more of physiological sensors including but not limited to temperature, O2 saturation, user feedback on level of tinnitus (ringing or noise) experienced.

In some instances by administering topically, at or near the auxiliary car canal a minimally invasive method is disclosed to add at least one of a vasodilator and a TNF-α inhibitor application of which may be via a predetermined spray dosage into the era canal or preferably via a preloaded swab packed in a disposable single use package. Administration via topical is achieve by adding one of a transdermal agent such as DMSO, Transfersomes™ or other transdermal delivery compounds for supplementing or treating auditory systems.

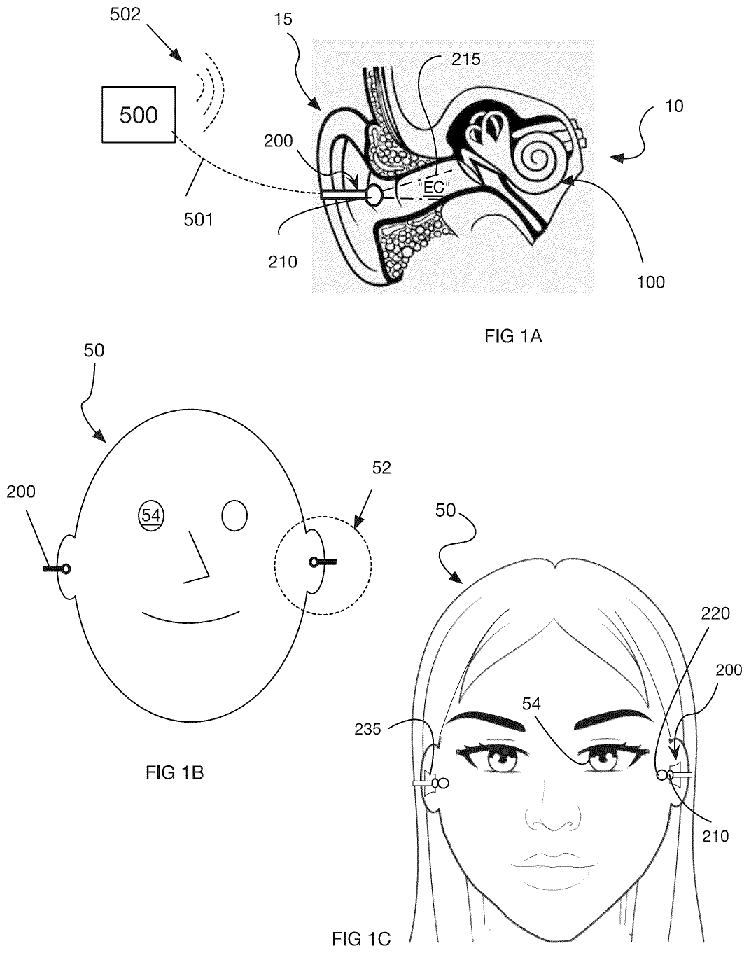

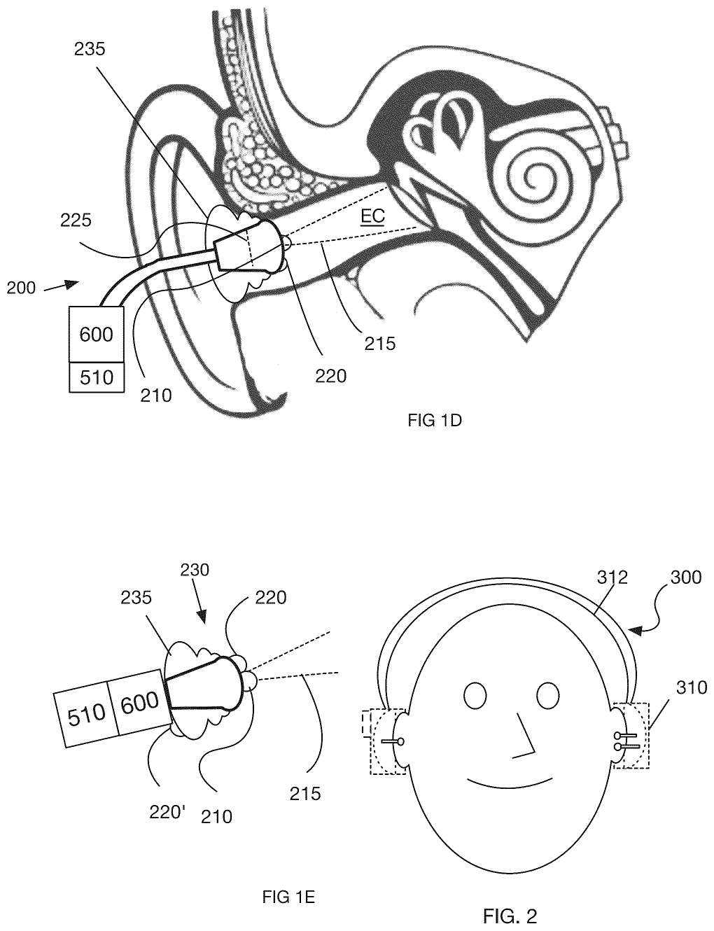

FIGS. 1A through 9B disclose nonlimiting aspects of compositions and devices as well as control systems and compliance schema. The compositions herein can be included in a pharmaceutical or nutraceutical composition together with additional active agents, carriers, vehicles, excipients, or auxiliary agents identifiable by a person skilled in the art upon reading of the present disclosure, and such compositions are within the scope of this disclosure. All publications cited herein are hereby incorporated by reference as if fully set forth herein.

The compositions disclosed herein can be included in a pharmaceutical or nutraceutical composition together with additional active agents, carriers, vehicles, excipients, or auxiliary agents identifiable by a person skilled in the art upon reading of the present disclosure, and such compositions are within the scope of this disclosure. All publications cited herein are hereby incorporated by reference as if fully set forth herein.

A pharmaceutically effective dose (ED) or effective concentration (EC) is a dose or concentration of an element (such as hydrogen), a phytochemical, compound or drug that produces a biological response. The term effective dose is used when measurements are taken in vivo, while the term effective concentration is used when the measurements are taken in vitro. This is generally defined by the range between the minimum effective dose (MED) and the maximum tolerated dose (MTD). The MED is defined as the lowest dose level of a pharmaceutical product that provides a clinically significant response in average efficacy, which is also statistically significantly superior to the response provided by the placebo. Similarly, the MTD is the highest possible but still tolerable dose level with respect to a pre-specified clinical limiting toxicity. In general, these limits refer to the average patient population.

An effective dose or aliquot of energy (EDE) or effective concentration of energy (ECE) of light (photons) or heat provided by such light to be bioactive, Bioactive includes but is not limited to the EDE or ECE to produce a biological response. The term EDE is used when measurements are taken in vivo, while the term effective concentration is used when the measurements are taken in vitro. This is generally defined by the range between the minimum effective energy dose (MEED) and the maximum tolerated dose (MTD).

In some instances a mammalian a subject to is treated via application of intravenously (IV) administered of gelatin with a 1000 ml Infusionslösung enthalten: Gelatinepolysuccinat 40,00 g (Mw 30 000, Mn 23 200) Elektrolyte: Natrium 154 mmol/l (3535 mg/l) Chlorid 120 mmol/l Sonstige Bestandteile 1000 ml Infusionslösung enthalten 154 mmol Natrium als Natriumchlorid. Along with between 7.5 and 15 grams of vitamin C in at least 250 cc of saline. They may be administered before or during application of the EDE. If a topical agent (as disclosed herein) is applied, the topical agent may be applied prior to the application of the EDE.

FIG. 1A is an overview of gross anatomy of an ear 10 having an outer wear 15. The ear canal “EC” is a pathway to the cochlea 100 which is a tiny, snail-shaped structure. It is the main organ of hearing and is part of the inner ear. Cochlear damage means that all or part of your inner ear has been hurt. Damage to the cochlea typically causes permanent hearing loss. This is called sensorineural hearing loss (SNHL). Many things can cause SNHL, or cochlear damage, including loud or extended noise exposure, certain powerful antibiotics, meningitis, Meniere's disease, acoustic tumors, and even the natural decline in age can cause hearing loss.

Noise-induced hearing loss, or NIHL, occurs when ears are exposed to overly loud sounds. Any sound over 85 decibels is considered to be dangerous to the ears and hearing. The louder the sound, the shorter the length of time that is considered “safe” exposure. For example, at a loud rock concert, sound may reach 115 decibels, and only a short 15 minutes can cause cochlear damage. In addition, repeated exposure to loud sounds can cause additional cochlear damage. The only way to prevent noise induced hearing loss is to limit exposure to loud sounds and wear car protection.

Shown in FIGS. 1A-3B are aspects of energy delivery devices 200 used with methods, therapies and devices disclosed herein to deliver at least an adjuvant and/or light energy/EDE to the ear canal “EC” in a head 50 and more specifically to car region 52 and the tissue surrounding the cochlea 100 including the ear canal “EC”. An in car flexible or conforming body 235 may be used to fit the device in the user's ear. The device 200 is connected to a power supply via a PCB (see FIG. 4), and is controlled via a microprocessor 500 and/or (the controller may be referred to as a control module) 510. The energy is delivered as at least one of bioactive light such as RBL and blue light or as a localized heat via IR light to heat tissue in the car surrounding the energy deliver device 200. LEDs or lasers energy produced by the energy deliver device 200 are effective light energy as they may be tailored to have narrow beam angles 215 when outputting energy one or more sensors 220 are supported on the delivery device. Sensors include but are not limited to sensors which measures a condition in the car canal such as temperature, pressure, O2 saturation, reflective light and is an input to a controller. Sensors also include but are not limited to sensors which measure decibels, vibration of the car canal structure, frequency and/or duration of sound or noise. In an optional earbud configuration a speaker 225 for audio may be added. When inserted in the car canal the device 200 is configured to deliver EDE to the cochlear region. In some instances the sensor 220 communicates with a controller 510 which it is in signal communication with either via a wired connection 501 or via wireless communication protocols. As is known in the art the control is configured to provide power to the device. In some instance the device in each car includes a power supply 600.

FIG. 1E illustrates a smart earplug 230. An in car flexible or conforming body 235 fits into the car canal and holds the earplug in place. The earplug will reduce sound and noise entering the car canal. However, some sound and noise can penetrate. The smart earplug has one or more sensors 220 in the car canal and one or more sensors 220′ facing the environment outside the car canal. The external sensor 220′ measures decibels, frequencies and vibration of noise or sound. The external sensor is in signal communication with a controller 510 and the controller receives inputs from sensors 220 and 220′ to initiate the application of an EDE to the ear canal “EC” if exterior sound exceeds a predetermined limit. A non-limiting example is when an external noise level exceeds the rating of the device to dampen or muffle said noise. The internal sensor(s) 220 can be used to measure condition in the car canal including but not limited to pressure, temperature, decibels, vibration, noise and the measurement(s) are provided as input to the controller and used to initiate application of an EDE if the measurement exceeds a threshold within the car canal. If the temperature in the car canal exceeds a threshold the internal sensor temperature measurement is used by the controller to reduce, modify frequency of, or stop the application of EDE.

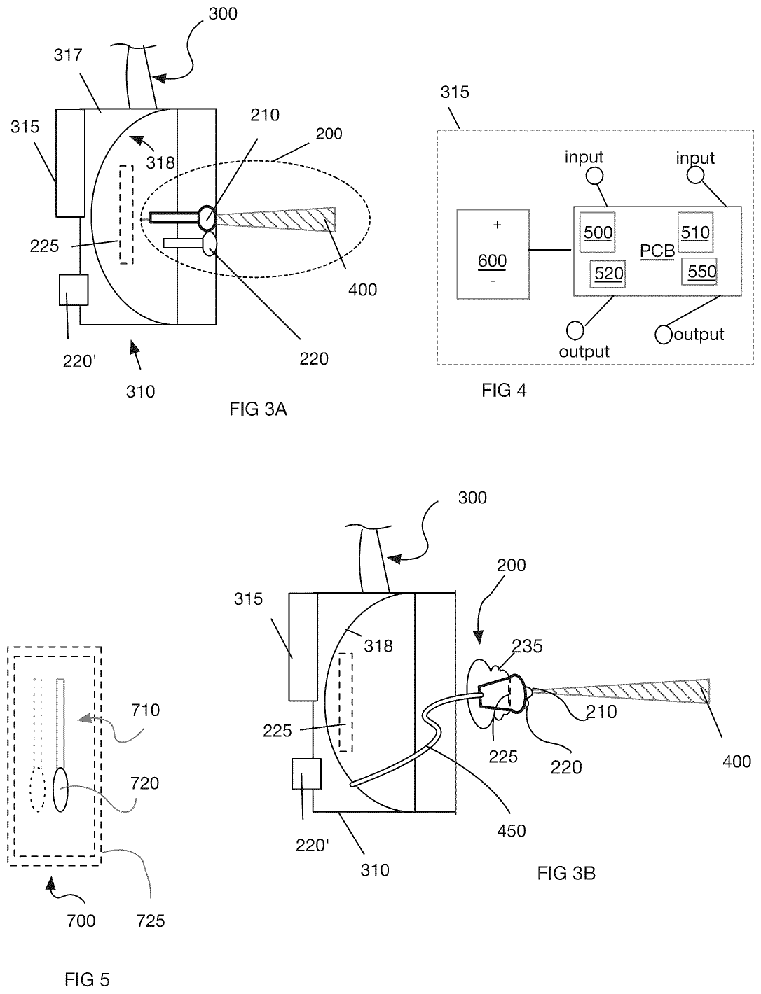

Shown in FIGS. 2 and 3A-3B show devices which incorporates energy delivery device(s) 200 as described above herein within over car wearable system 300. The wearable system is at least one of self-contained and powered via batteries or it may be a wired system that required wall outlet power and is not as portable. In some instances it includes at least one ear covering 310 is placed over the outer wear 15. In some instance the energy deliver device 200 is integrated with the inside 318 of the car coverings 317. The ear covering may be attached to a support member 312. There may be bilateral ear coverings connected to the same support member. Within the ear covering is an inside chamber 318 formed to at least one of position the energy delivery device 200 and position one or more sensor 220 and reflect scattered or reflected energy via a parabolic reflector back into the ear canal “EC”. The LED or laser will either have an integrated lens to produce a fixed beam angle 400 or one or more lenses, diffusers or light shaping filters may be added to achieve beam contouring. Such beam shaping systems and materials are known in the art and those of ordinary skill in the art will recognize that this disclosure teaches directing energy into the car canal and using known devices to focus or direct such energy is within the scope of this disclosure. The PCB and power supply 315 are shown attached to an car covering. An external sensor 220′ may be attached to the outside of the device to one of measure decibels, frequencies and vibration of noise or sound. The external sensor is in signal communication with a controller and the controller can use measured vibration, sound and/or noise to initiate the application of an EDE to the ear canal “EC” if exterior sound exceeds a predetermined limit. A non-limiting example is when an external noise level exceeds the rating of the device to muffle said noise. An internal sensor 220 (see FIG. 1D) measure provided as an input to the controller may be used to initiate application of an EDE if the sound noise, vibration or frequencies within the ear canal exceed a predetermine level. Including sound from a speaker 225 in the device 200 or 300. In some instances the energy delver device is attached to the inside chamber 318 via an electrical and/or data line in signal communication with the controller. The flexible line(s) 450 allows the user to insert the energy deliver device 200 (which also may be noise cancelling) into an ear covering which may be noise cancelling or reducing.

Moreover, in addition to using a red or long red mode to encourage vasodilation and nitric oxide “NO” production disclosed is a device also having a blue light mode which generates circadian stimulating energy and the ability to shift from a first mode to a second mode allows for both treatment and protection of hearing (visa vie ameliorating the effect of loud noise exposure (such as loud music or explosions)) and production of circadian stimulating blue light in an EDE to combat SAD and/or generally enhance at least one of memory, focus and concentration levels in subjects ID. Bright light therapy has been studied in association with ear canals but was a gross application of bright full spectrum light.

The energy deliver LED or lasers are envisioned as one or more at preselected wavelengths. RBL and blue light as well as IR (for heat generation) for example which penetrate the ear. The photodynamic aspects cause at least increased NO is helpful for vasodilation and to allow greater blood flow into the cochlear microvasculature. The system may be operated in multiple modes wherein the LEDs or lasers may simultaneously or sequentially provide specific wavelengths of light such as shifting from circadian stimulating blue (first mode) to RBL (second mode) and/or also to a third warming or heat mode. In addition an input device (not shown) such as a smart phone or tactile pad can be used by to communicate with the controller and increase or decrease any one of the heat and bioactive light corresponding to one of comfort and level of tinnitus (ringing). Via the input a user may set limits on the controller in the controller memory to control the heat, as measured by sensors, to control user comfort, alertness or other imputable user data.

FIG. 4 shows aspects of basic elements of the PCB and power supply 315 including but not limited to inputs and outputs. Inputs from sensors and outputs to the energy providing devices. A microprocessor 500 may the or may be used in conjunction with a controller 510, which may include PWM control. The user may specify whether the light energy temperature feels (is perceived by the user) to be too great or needs the temperature (as perceived by the user) may be adjusted to a higher temperature. The controller stores inputs and the setting can be used to control the light energy. 520 is included. Preferably a wireless protocol chip set 550 with antennae is included to allow communication and/or data transfer from the device to servers (see FIG. 7) which may be through a network.

FIG. 5 shows a disposable delivery package 700 wherein a barrier 725 contains a swab 710 having a wet end which is contains a predetermined single use dosage of DMSO (or other agent that is used in dermal deliver) and at least one of a vasodilator and a TNF-α inhibitor (such as Etanercept). In Tinnitus we have found that both vasodilation and inhibition of TNF-α show improvements in reducing the symptomology of tinnitus. Etanercept is extremely expensive and by applying it locally with DMSO very small aliquots of the Etanercept may form an effective dose for localized TNF-α inhibition.

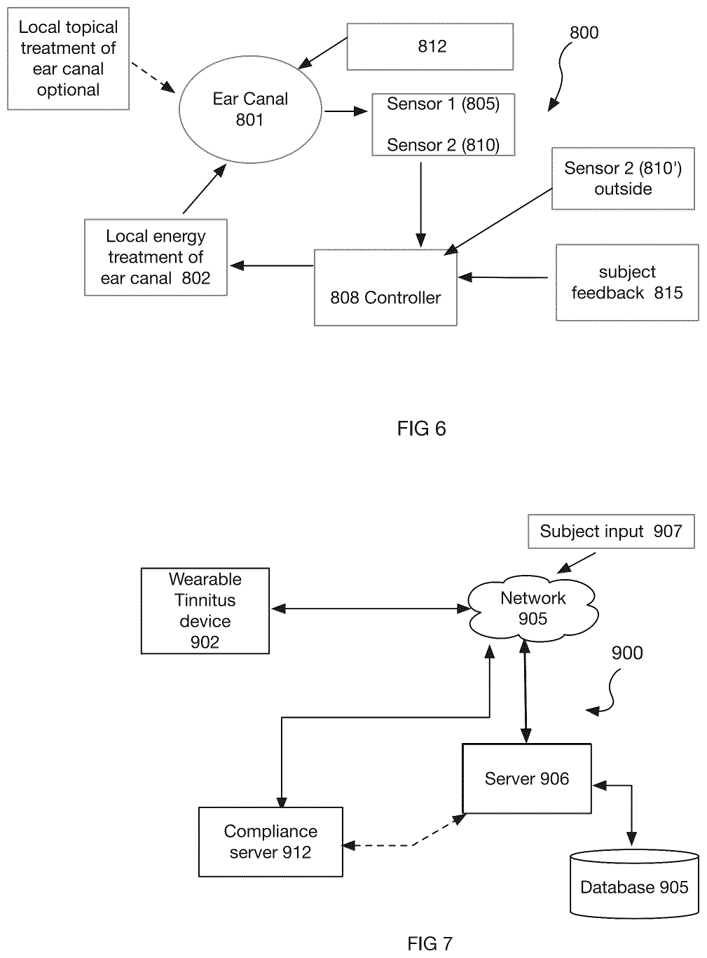

FIG. 6 is an overview of the portion of the method of treatment which may include combinatorial therapy for the energy delivery device 800. The delivery of light spectra in an EDE or ECE to the era canal is disclosed. The delivery may be as a supplement to maintain hearing homeostasis or health akin to a vitamin or mineral supplement. In other instances it may be to mitigate the impact of loud noise or music. In some instance it relieves tinnitus symptoms. In some instance it is protective of cochlear microvasculature. Said EDE in one or more modes may be applied to adjust circadian cycles, increase concentration and protect hearing. Modes refer to one more of different light spectra and different durations and intensity of EDE applied. In some instances the EDE may be combined with a treatment including Topical Treatment of Ear Canal and an IV drip for more acute cases of tinnitus.

An energy deliver device 802 is placed in photonic contact with the car canal. Photonic contact means it is position to deliver light energy to the car canal and structures therein. The degree to which the light penetrates tissue and structures in the car canal. Penetration also depends in part in on the spectrum of the light (wavelength) and duration as well as positioning of the applied light and intensity. A first sensor or group of sensors 805, in signal communication with a controller 808 and measures a condition in the car canal such as temperature, pressure, O2 saturation, reflective light. The sensor 805 provides an input to the controller wherein according to presets or dynamic adjustment if a threshold level is exceeded or met the controller adjusts the application of EDE including but not limited to turns on or off the application of light energy, reduces the intensity (joules) of light energy, alters the wavelength and modifies the frequency of the light energy.

A second sensor or group of sensors 810, in signal communication with a controller, measures one or more of decibels, vibration of the car canal structure, frequency and/or duration of sound or noise and/or the measures decibels, vibration of the car canal structure. A third group of sensor(s) 810′ measure one or more of decibels, vibration, frequency and/or duration of sound or noise outside of the car canal in the environment and the controller turns on or off the application of light spectrum energy (the EDE) if a measured sound level is below, at, or exceeds a threshold. The threshold may be fixed, preset at different levels based on noise levels, set as defined by a user via a user interface, or variable based on measured levels of at least one of noise, vibration, decibels, and may include duration of the measured level as one of the threshold level monitored. Additional sounds such as music 812 in ether the mode of playing music through the device or being in a music environment such as a concert or theater which impact the user and the car canal can also be input levels for the device which may trigger the controller to administer the light spectral energy (or EDE) in response to the monitored levels and threshold(s) exceeded or met.

For a non-limiting example, if the user is playing music via a PMP with speakers into the car canal and the output is at a level which exceeds a threshold the device controller is configured to dynamically adjust the EDE to a level to support cochlear microvascular health when noise stress is directed at the cochlea. In other instances a time clock in the controller may also adjust circadian stimulating (blue) lighting in the car canal or red, long red and near IR (BRL) in the car canal to reset or support healthy circadian cycles. User feedback 815 to the controller from known methods which include but are not limited to spoken commands, touch pad, computing device Apps and the like can also be used to supply subject feedback for additional adjustment of the system.

For a non-limiting example, if the user is taking/applying an EDE supplement the controller is configured to provide the EDE during a pre-determined time subject to sensor measurements such as temperature which exceed a threshold.

In some instances as a supplement to maintain hearing health a user may initiate the system to have the controller 808 deliver EDE based on a preset time and subject to safety threshold measurements (which would turn down intensity or off the application of EDE) such as temperature in the car canal.

Optionally, the intravenously (IV) administered compound include gelatin with a 1000 ml Infusionslösung enthalten: Gelatinepolysuccinat 40,00 g (Mw 30 000, Mn 23 200) Elektrolyte: Natrium 154 mmol/l (3535 mg/l) Chlorid 120 mmol/l Sonstige Bestandteile 1000 ml Infusionslösung enthalten 154 mmol Natrium als Natriumchlorid. Along with between 7.5 and 15 grams of vitamin C in at least 250 cc of saline may be administered before or during application of the EDE. If a topical agent (as disclosed herein) is applied, the topical agent may be applied prior to the application of the EDE. One or more sensors may supply data such as O2 levels and temperature within at least one ear covering. That data is processed by the controller and sued to adjust the energy delivered.

In therapeutic treatment compliance and monitoring compliance is often a challenge. Accordingly, in some instances the Wearable Tinnitus Device (see FIG. 7) utilizing the EDE applied to the car canal as previously describer may be operated generally under the protocol set forth in FIG. 7. The Wearable Tinnitus Device (WTD) 902 is placed in signal communication with a network 905. The network communicates with a server 906 cand provides data either from memory stored on the WTD (as described previously) or real time use data. Data may include time of use, duration of use, date of sue, any user feedback 907 adjustments and the like. The server is in signal communication with a database 909 which may store the collected data. The network or the Server may also communicate with a compliance server 912 wherein records of use by the user may be maintained. Such records are critical to agglomerate to determine if the user (if use is remote from a treatment provider) is actually using the device for sufficient time and regularly and how that use corresponds to level of discomfort The data which may be transmitted is at least all data described within. The user of the WTD may input feedback 907 during treatment as to whether symptoms have changed during treatment, whether the light energy temperature feels (is perceived by the user) to be too great or needs the temperature (as perceived by the user) may be adjusted to a higher temperature. The controller stores inputs and the setting can be used to control the light energy.

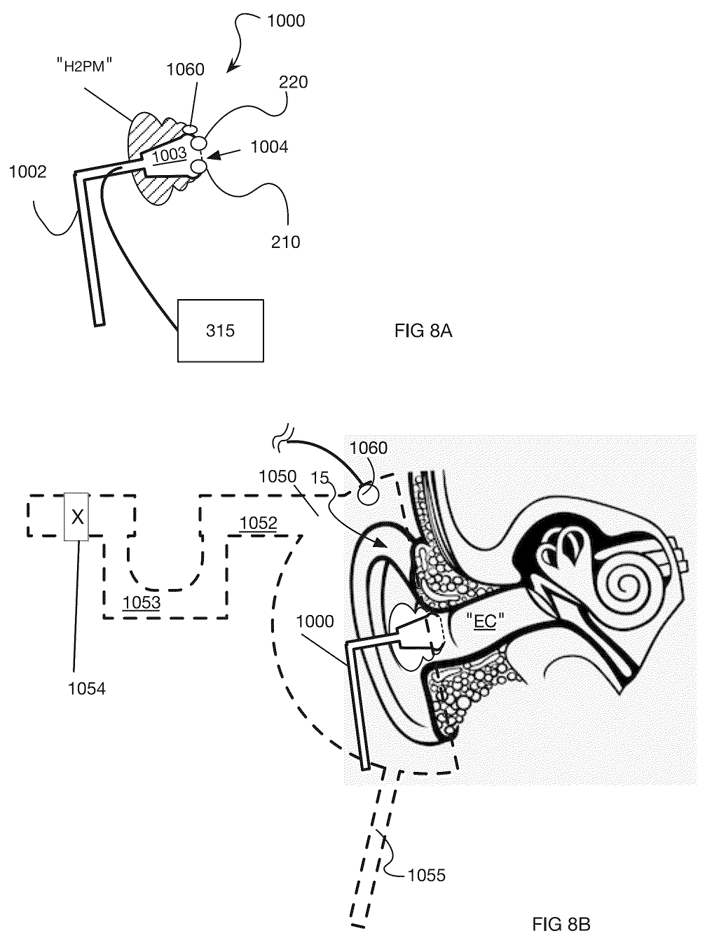

Shown in FIGS. 8A and 8B are aspects of energy and fluid delivery device 1000 used with methods, therapies and devices disclosed herein to deliver at least an adjuvant and/or EDE to the tissue surrounding the cochlea 100. The device 1000 is connected to a power supply via a PCB 315 and is controlled via a microprocessor controller 500 the LED or laser 210 EDE as described above photonic energy is delivered as at least one of bioactive light such as RBL and blue light or as a localized heat via IR light to heat tissue in the car canal surrounding the energy deliver device 1000. LEDs or lasers are effective devices as they may be tailored to have narrow beam angles when outputting energy and sensors 220 as described above may be attached.

The wearable system 1000 is at least one of self-contained. In its simplest iteration the equivalent of an in-car headphone or “earbud” is the ear canal “EC”. A hydrogen permeable outer shell “H2PM” forms a cavity 1003 which is fluidly connected 1002 to a source for at least on of oxyhydrogen fluid and hydrogen fluid and provides a fluid pathway through an end nozzle or aperture 1004 to form a passageway for hydrogen or oxyhydrogen to pass into the car canal. The hydrogen permeable outer shell or permeable membrane H2PM is configured to position the device in the car canal to support gaseous fluid flow and to at least one of release from any seal formed seal with the car canal and allow gaseous fluid above a preselected pressure in the car canal to vent out of the car canal. In some optional instances, a hydrogen sensor 1060 in signal communication with a controller whereby the quantity of gaseous hydrogen measured may be used at the controller to adjust the flow of hydrogen of oxyhydrogen to the device. In some optional instances the device and method may also include the aforementioned means to deliver aliquots of energy via LEDs or lasers 210 powered via batteries or it may be a wired system that required wall outlet power and is not as portable. Sensors 220 to measure conditions in the ear canal as previously described may be included. In some instances a collection cover 1050 may be placed over the ear whereby gaseous fluid exiting the ear canal may be vented through a fluid connection 1052 and a hydrogen sensor 1060 in signal communication with a controller whereby the quantity of gaseous hydrogen measured may be used at the controller to increase or decrease the flow of hydrogen of oxyhydrogen to the device may be measured in the collection cover 1050. Said collection cover provides a pathway to direct the spent hydrogen gas or spent oxyhydrogen for disposal.

In other instances an optional inlet 1055 for gaseous fluid such as hydrogen or oxyhydrogen may directly feed gaseous fluid into the collection cover 1052. In this exemplar the wearable system may be removed and the gaseous fluid supply to the ear canal is provided via the collection cover via the input 1055 and the collection cover is configured to partially seal around the car. The fluid collection may have a trap 1053 whereby the lighter than air hydrogen will flow out of the device when it fills the trap. Alternatively a one way valve 1054 may be configured to allow passage of gaseous fluid above a predetermined pressure. In some optional instances, a hydrogen sensor 1060 in signal communication with a controller whereby the quantity of gaseous hydrogen measured may be used at the controller to adjust the flow of hydrogen of oxyhydrogen to the device.

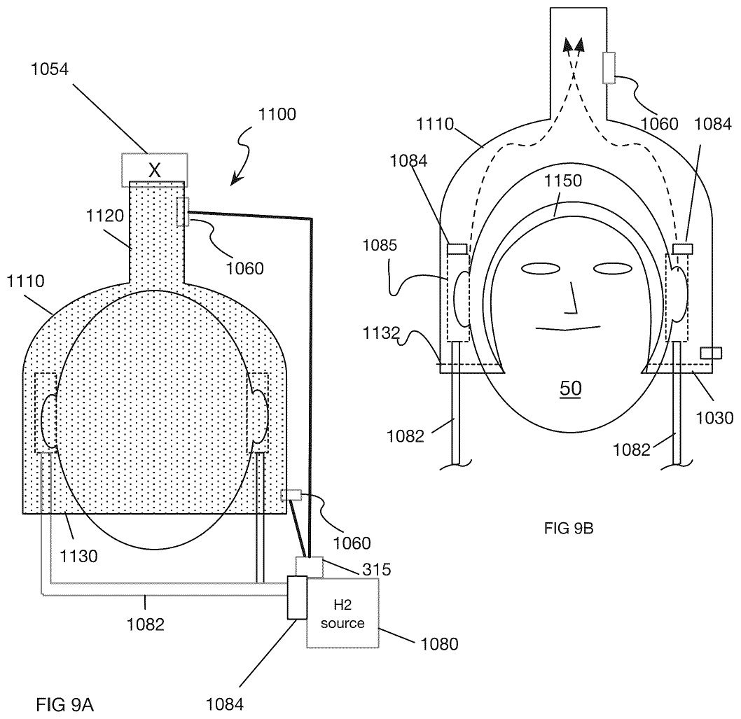

FIGS. 9A-9B show a series of exemplary implementation wherein an enclosure covers the ears 10 of the subject.

This disclosure provides novel devices, systems and methods to bath the ears and inner ear structure with hydrogen rich gaseous fluid, this is a local application which does not require digestion of water laced with hydrogen or inhalation of hydrogen gas. Hydrogen aliquots are supplied to and permeate the tissues and structures.

Age-related hearing loss (ARHL) or presbycusis is the third most prevalent chronic disease among older adults. It can vary in severity from mild to severe, the more severe forms affecting communication and contributing to social isolation, depression, and possibly dementia. A recent study exposed an animal model to high levels of H₂O₂ and it has been shown that the H2O2-exposed cochlear cells exhibited several additional senescence-associated properties, including high senescence associated β-galactosidase activity mainly in sensory hair cells, together with increased levels of p21, p38, and p-p38. The in vivo results show senescence-accelerated premature cochlear aging. Menardo J, Tang Y, Ladrech S, Lenoir M, Casas F, Michel C, Bourien J, Ruel J et al (2012) Oxidative stress, inflammation, and autophagic stress as the key mechanisms of premature age-related hearing loss in SAMP8 mouse cochlea. Antioxid Redox Signal 16 (3): 263-274. https://doi.org/10.1089/ars.2011.4037. Mitigation of ROS on H2O2-induced DNA damage and senescence-like phenotype, with 10 μM EUK-207, a potent synthetic superoxide dismutase/catalase mimetic that scavenges superoxide and hydrogenperoxide and the study reported that (i) ROS is connected to age-related sensory hair cell degeneration, (ii) ROS-induced DDR driving senescence-like features may account for premature cochlear aging, and (iii) pharmacological scavenging of superoxide and hydrogen peroxide can mitigate. (Benkafadar, N., François, F., Affortit, C. et al. ROS-Induced Activation of DNA Damage Responses Drives Senescence-Like State in Postmitotic Cochlear Cells: Implication for Hearing Preservation. Mol Neurobiol 56, 5950-5969 (2019). https://doi.org/10.1007/s12035-019-1493-6)

Hydrogen inhalation and hydrogen infused water has been shown in animal models to have some ROS mitigation. Huang, C. S., Kawamura, T., Toyoda, Y., and Nakao, A. (2010). Recent advances in hydrogen research as a therapeutic medical gas. Free Radic. Res. 44, 971-982.doi: 10.3109/10715762.2010.5

Hydrogen has been shown to have no adverse effects and great efficacy on nearly all pathogenic states involved in oxidative stress and inflammation. Ohta, Shigeo. (2014). Molecular hydrogen as a preventive and therapeutic medical gas: Initiation, development and potential of hydrogen medicine. Pharmacology & therapeutics. 144. 10.1016/j.pharmthera.2014.04.006

One exemplary system 1100 covering the head 50 of a subject may optionally have hydrogen sensors 1060 configured to measure hydrogen within the enclosure. The enclosure has a body 1110 fluidly connected to a vent 1120 and an may have a partially opened bottom 1130 to allow atmospheric air to pass in a flow restrictive valve 1054 may be added at the end of the vent to control gaseous fluid flow out of the enclosure. Said vent in some instances may be connected to a source of negative pressure (not shown). A source of hydrogen 1080 which may be a tank, a membrane electrolyzer, a generator or the like is fluidly connected 1082 to ear covering 1085. The hydrogen source specifically includes but is not limited to a source for oxyhydrogen (HHO) such as a generator or tank storage. The connection may be controlled by a flow restricting means such as a valve 1084 to control the pressure and quantity of gaseous fluid delivered. The valve may be controlled by a controller 315. The controller may receive input from optional pressure sensors 1060 to adjust the gaseous fluid flow. In some instance the bottom 1130 is at least partially sealed along the line of 1032.

The ear coverings are configured to cover the outer ear and direct gaseous flow into the ear canal. The ear covering does not need to seal over the ears. The coverings may also be permeable to hydrogen at least one of a predetermined rate and pressure to allow hydrogen to pass therethrough. In some instances a valve or flow control means 1084 may be in fluid connection with the ear covering whereby gaseous fluid within the ear covering may be controllable released into the enclosure. Restoration of hearing or reduction of hearing losses during the ageing process can ameliorate cognitive decline related to loss of hearing.

What is claimed is:

1. A device to deliver therapeutically effective light energy to at least increase circulation in cochlear microvasculature, the device comprising:
an energy delivery device comprising;
a means to emit light energy;
a power supply;
one or more temperature sensors in thermal communication with the ear canal;
a microprocessor controller in signal communication with the means to emit light energy, the power supply and the one or more temperature sensors;
wherein the controller is configured to deliver at least one effective dose or aliquot of light energy into an ear canal to produce heat in the ear canal; and,
wherein the controller is configured to receive temperature sensor input and to control outputs of energy to limit heat in the ear canal.

2. The device of claim 1 wherein the controller is configured to receive a user's selected comfort level input to limit heat in the ear canal.

3. The device of claim 1 wherein said effective dose one or more of increases circulation in the cochlea vasculature, increases vasodilation in the cochlear microvasculature and increases nitric oxide in the local area adjacent to the cochlea.

4. The device of claim 1 wherein the effective dose is at least one of red light, long red light, near IR light and blue light.

5. The device of claim 1 wherein the light is delivered via sequential modes adjusted by the control module.

6. The device of claim 2 wherein the controller is configured to adjust of mode, frequency, duration and intensity of the effective dose by way of sensor input to the control module.

7. The device of claim 1 further comprising:
the controller receives sensor input of one or more of decibels, vibration of the ear canal structure, frequency and/or duration of sound or noise; and,
the controller decides if a threshold is met for application of the dose or aliquot of light energy.

8. The device of claim 1 further comprising:
the controller receives sensor input of one or more of decibels, vibration, frequency and/or duration of sound or noise outside the ear canal; and,
the controller decides if a threshold is met for application of the dose or aliquot of light energy.

9. The device of claim 7 wherein one or more sensors provide input to the controller for adjustment of mode, frequency, duration and intensity of the effective dose based on the frequency and/or duration of sound or noise outside the ear canal.

10. The device of claim 1 wherein the energy delivery device is configured to fit in an over the ear chamber and said chamber positions the light output into the ear canal.

11. The device of claim 10 wherein the energy delivery device is configured to be connected by flexible lines in the over the ear chamber.

12. A system to deliver therapeutically effective light energy to at least the cochlear microvasculature, the system comprising:
an energy delivery device configured to fit into the ear canal of a user having a controller in signal communication with at least a means to emit light energy, a power supply, one or more temperature sensors in thermal communication with the ear canal;
wherein the controller is configured to cause the energy delivery device to deliver at least one effective dose or aliquot of light energy into an ear canal; and,
wherein the controller is configured to receive temperature sensor input and to control outputs of energy to limit heat in the ear canal.

13. The system to deliver therapeutically effective light energy to at least the cochlear Microvasculature of claim 12 wherein the controller is configured to receive sensor input of one or more of decibels, vibration of the ear canal structure, frequency and/or duration of sound or noise; and,
the controller decides if a threshold level of one or more of decibels, vibration of the ear canal structure, frequency and/or duration of sound or noise is met.

14. The system to deliver therapeutically effective light energy to at least the cochlear microvasculature, of claim 13 wherein if the threshold level is met the controller causes the application of the dose or aliquot of light energy.

15. The system to deliver therapeutically effective light energy to at least the cochlear microvasculature, of claim 14 wherein the controller is configured to receive a user's selected comfort level input to limit heat in the ear canal; and,
if the sensor input exceeds the user input threshold of heat, the controller reduces the application of the dose or aliquot of light energy
